(19) United States
(12) Patent Application Publication
(10) Pub. No.: US 2009/0036796 A1
(43) Pub. Date: Feb. 5, 2009

(54) Liquid Sampling Apparatus and Method of Using Same

(76) Inventors: Peter E. Rising, Brightwaters, NY (US); Brian H. Rutledge, Eldersburg, MD (US)

Correspondence Address: F. CHAU & ASSOCIATES, LLC, 130 WOODBURY ROAD, WOODBURY, NY, 11797, US

(21) Appl. No.: 12/278,200

(22) PCT Filed: Feb. 5, 2007
(86) PCT No.: PCT/US07/03047

§ 371 Date: Aug. 4, 2008

Related U.S. Application Data
(60) Provisional application No. 60/764,956, filed on Feb. 3, 2006.

(51) Int. Cl. A61B 5/00; G01N 33/50

(52) U.S. Cl. 600/573; 422/68.1

(57) ABSTRACT

A sampling apparatus including an ampoule, an ampoule barrel for receiving the ampoule through a first opening, and a sheath fitted over a tip of the ampoule and extending from the ampoule and through a second opening of the ampoule barrel.

### CROSS-REFERENCE TO RELATED APPLICATIONS

This application claims priority to U.S. Provisional Application Ser. No. 60/764,956, filed on Feb. 3, 2006, which is herein incorporated by reference in its entirety. Further, this is a continuation-in-part application claiming priority to U.S. application Ser. No. 11/074,466, filed on Mar. 7, 2005, which is herein incorporated by reference in its entirety.

### BACKGROUND OF THE INVENTION

1. Technical Field

The present invention relates to sampling liquids, and more particularly, to an apparatus for sampling a liquid and automatically performing a test.

2. Discussion of Related Art

Sampling methods for liquids typically involve drawing a sample into a pipet, syringe, or other container from a cup. Such a sampling method exposes the sample taker to the liquid. Limiting a sample taker's exposure to a sample may be desirable, such as in urine analysis. Further, sampling methods may include exposing the sample to contaminants leading to, for example, sampling errors.

Therefore, a need exists at least for a system and/or method for reducing exposure to a sample, reducing sampling errors and limiting contamination of test samples.

### SUMMARY OF THE INVENTION

According to an embodiment of the present disclosure, A sampling apparatus including an ampoule, an ampoule barrel for receiving the ampoule through a first opening, and a sheath fitted over a tip of the ampoule and extending from the ampoule and through a second opening of the ampoule barrel.

### BRIEF DESCRIPTION OF THE DRAWINGS

Preferred embodiments of the present invention will be described below in more detail, with reference to the accompanying drawings:

FIGS. 1A-C are an illustration of a test system according to an embodiment of the present disclosure;

FIG. 2 is a cross-section view of the test system of FIG. 1;

FIG. 3 is a cross-section view of a tip area of the test system of FIG. 1;

FIGS. 4A-C are illustrations of a sampling cap, sheath and ampoule according to an embodiment of the present disclosure;

FIGS. 5A-D are illustrations of an ampoule barrel having no offset according to an embodiment of the present disclosure;

FIGS. 6A-D are illustrations of an ampoule barrel having an offset according to an embodiment of the present disclosure;

FIGS. 7A-D are illustrations of an ampoule barrel having an offset according to an embodiment of the present disclosure;

FIGS. 8A-C are illustrations of a carrier for a test system according to an embodiment of the present disclosure;

FIGS. 9A-C are illustrations of a breaking mechanism according to an embodiment of the present disclosure;

FIG. 10 is a flow chart of a method according to an embodiment of the present disclosure; and

FIGS. 11A-B are illustrations of a test system according to an embodiment of the present disclosure.

### DETAILED DESCRIPTION OF PREFERRED EMBODIMENTS

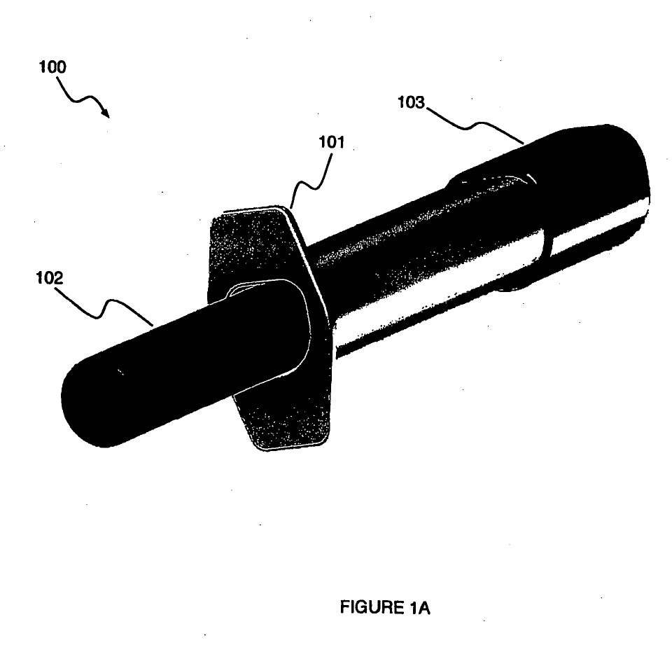

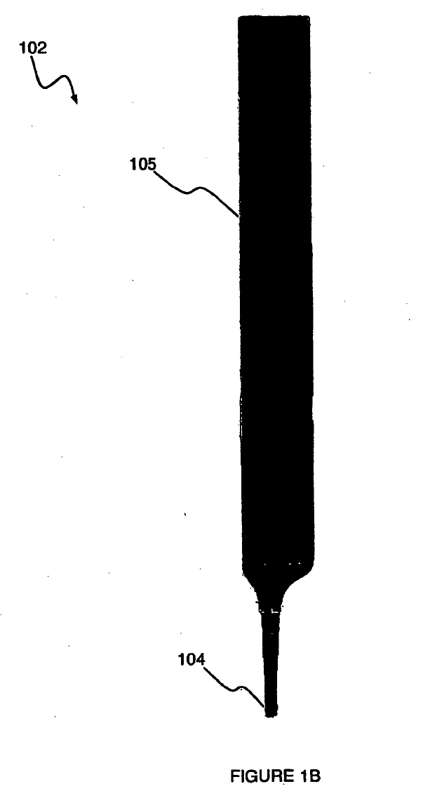

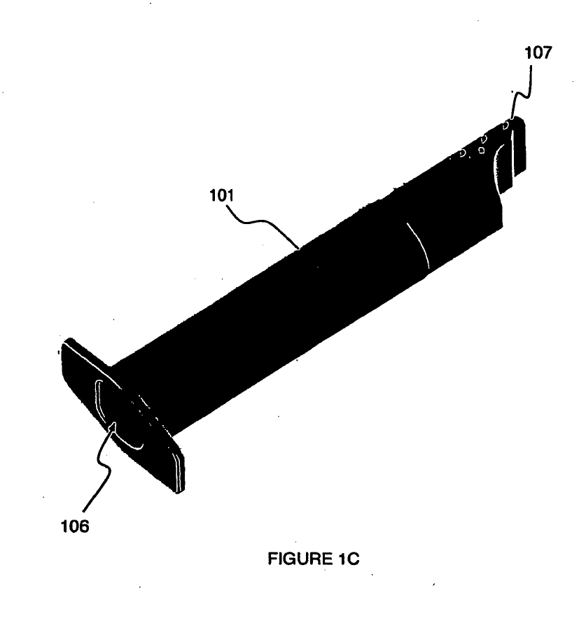

A test system according to an embodiment of the present disclosure is a self-contained total microbe test Referring to FIG. 1A, a test system 100 includes an ampoule barrel 101, an ampoule 102 and a cap 103. The ampoule 102 may be removed from the ampoule barrel 101.

Referring to FIG. 1B, the ampoule 102 includes a frangible tip 104. The frangible tip 104 may be offset from a center of a main body 105 of the ampoule 102. The ampoule 102 is a sealed container, which is opened open breaking the frangible tip 104. The interior of the ampoule 102 is a vacuum-sealed environment. Upon, breaking the tip 104, the ampoule 102 is adapted to draw a predetermined volume of liquid according to an internal pressure of the ampoule 102.

Referring to FIG. 1C, the ampoule barrel 101 includes an opening 106 for receiving an ampoule. The ampoule barrel 101 includes a tip 107 for allowing a liquid into the ampoule barrel 101. The barrel tip 107 includes one or more ports for drawing the liquid into the ampoule barrel 101 under the vacuum of an ampoule. The tip 107 of the ampoule barrel may be off center relative to a longitudinal center of the ampoule barrel 101. An off center tip 107 may be inserted into a sampling cup, wherein the sampling cup may be held on edge to pool a sample, increasing a depth of the sample. The off center tip 107 may be immersed in the pooled sample.

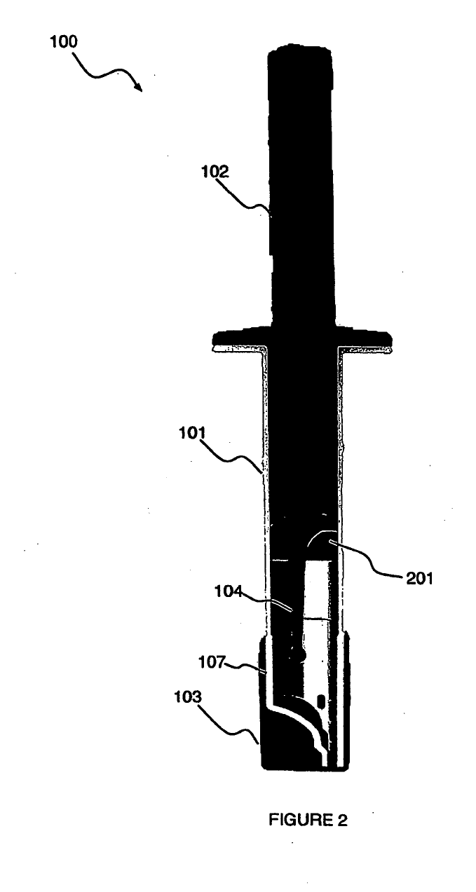

FIG. 2 is a cross-section view of a test system 100. An ampoule barrel 101 receives an ampoule 102. A seal 201, formed of for example, a rubber substance secures the ampoule 102. A tip 104 of the ampoule 102 projects through the seal 201 and into a sample chamber. Upon placing the barrel tip 107 in a liquid, the seal 201 prevents the liquid from passing into an upper portion of the ampoule barrel 101. The cap 103 is removable. The cap 103 prevents contaminants from entering the ampoule barrel 101 or contacting the barrel tip 107 prior to a test.

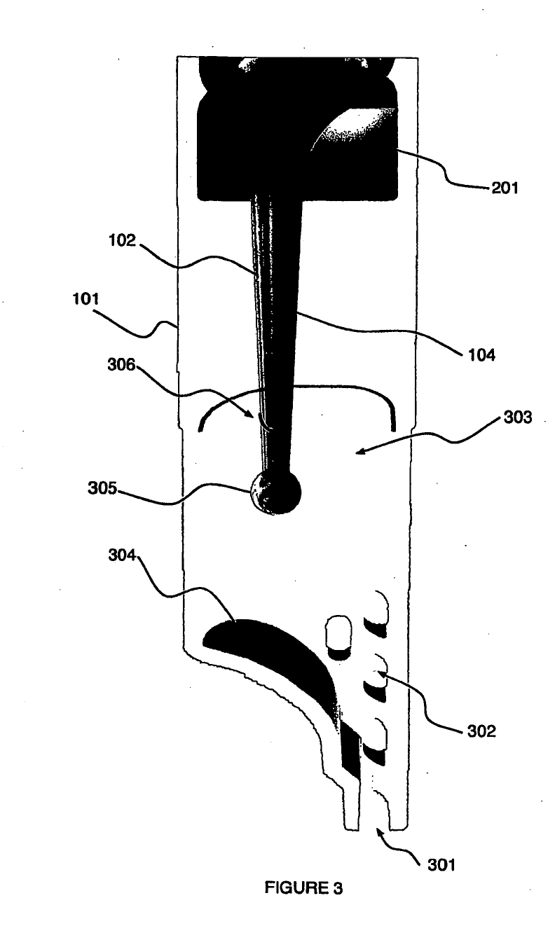

Referring to FIG. 3, the ampoule barrel 101 includes ports, e.g., 301 and 302, for allowing a liquid to pass into a lower portion or sample chamber 303 of the ampoule barrel 101. The ports may be located on an end, e.g., 301, of the ampoule barrel 101 and/or on a side of the ampoule barrel, e.g., 302. The seal 201 prevents the liquid form passing into an upper portion of the ampoule barrel 101. The ampoule 102 includes a frangible tip 104. The tip 104 includes a terminus 305. The terminus 305 has a rounded structure. Above the terminus 305, a score 306 is provided on a portion of the tip 104. The score 306 is located to control the height at which the tip 104 breaks. The score 306 may be located on a portion, e.g., about 90 degrees or about 180 degrees, of the circumference of the tip 104. For example, as shown in FIG. 3, the score 306 is located on a portion of the tip 104 away from the direction of the intended breakage. The ampoule 102 may be depressed into the ampoule barrel 101, wherein the terminus 305 meets an angled face 304 of the ampoule barrel 101. A pressure applied to the ampoule 102 causes the ampoule to descend into the ampoule barrel 101 with the seal 201. The angled face 304 converts the pressure into a lateral pressure on the terminus 305 of the tip 104. The tip 104 breaks at about the height of the score 306 under the lateral pressure. The terminus 305 breaks away from the tip 104 allowing the liquid in the lower portion 303 of the ampoule barrel 101 into the ampoule 102. A flow rate at which the ampoule 102 draws liquid may be slower than a flow rate of the ports of the ampoule barrel 101. The tip 104 may be sheathed, such that upon breaking the terminus 305, a lower portion of the tip drop away into the ampoule barrel 101 and a remaining portion of the tip 104 is guarded within the sheath.

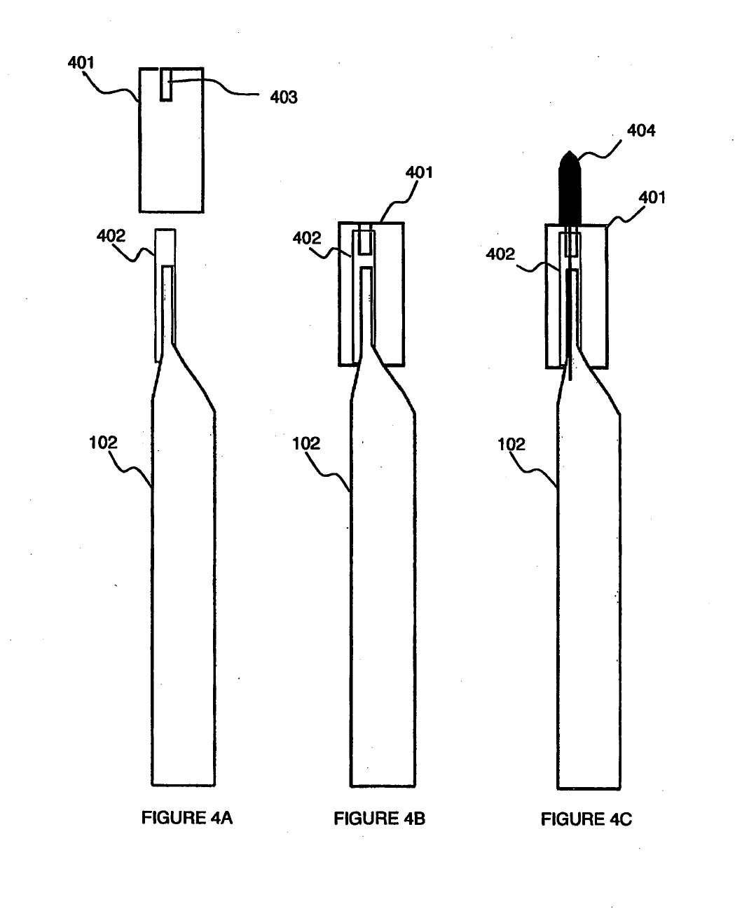

Referring to FIGS. 4A-C, upon drawing a sample into the ampoule 102 a sampling cap 401 may be placed over a broken tip of the ampoule 102. A sheath 402 guards any sharps. The sampling cap 401 includes a nipple 403. The nipple 403 fits within the sheath 402. The cap 401 reduces a potential for contact with the liquid in the ampoule 102. The nipple 403 cooperates with the sheath 402, securing the sampling cap 401 to the ampoule 102. The nipple 403 may be a tube through which a syringe 404 or other device may gain access to the contents of the ampoule 102.

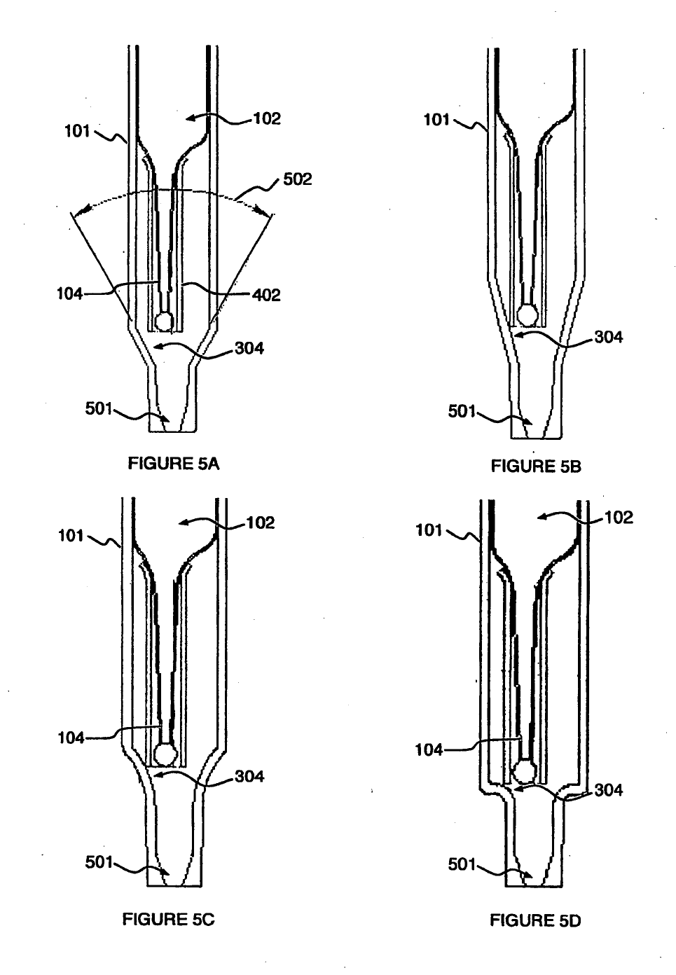

Referring to FIGS. 5-7, various ampoule barrels are depicted. It should be noted that modifications and variations of the ampoule barrels are contemplated herein.

Referring to FIGS. 5A-D, ampoule barrels 101 having a bottom port 501 with no offset relative to a centerline of the ampoule barrel 101 are shown. Referring to FIG. 5A, a lead in angle 502 of the face 304 is about 60 degrees. The ampoule 102 includes an offset tip 104. The tip 104 is guarded by a sheath 402. The offset tip 104 is aligned to meet the face 304 as the ampoule 102 descends into the ampoule barrel 101. FIG. 5B illustrates an ampoule barrel 101 having a face 304 with a lead in angle of about 30 degrees. FIGS. 5C and 5D illustrate ampoule barrels 101 having a face 304. The face 304 has a radius. For example, the radius of the face 304 in FIG. 5C is about 0.500 inches and the radius of the face 304 in FIG. 5D is about 0.110 inches. The radius of the face 304 converts the descent of the ampoule 102 into a lateral force that breaks the tip 104.

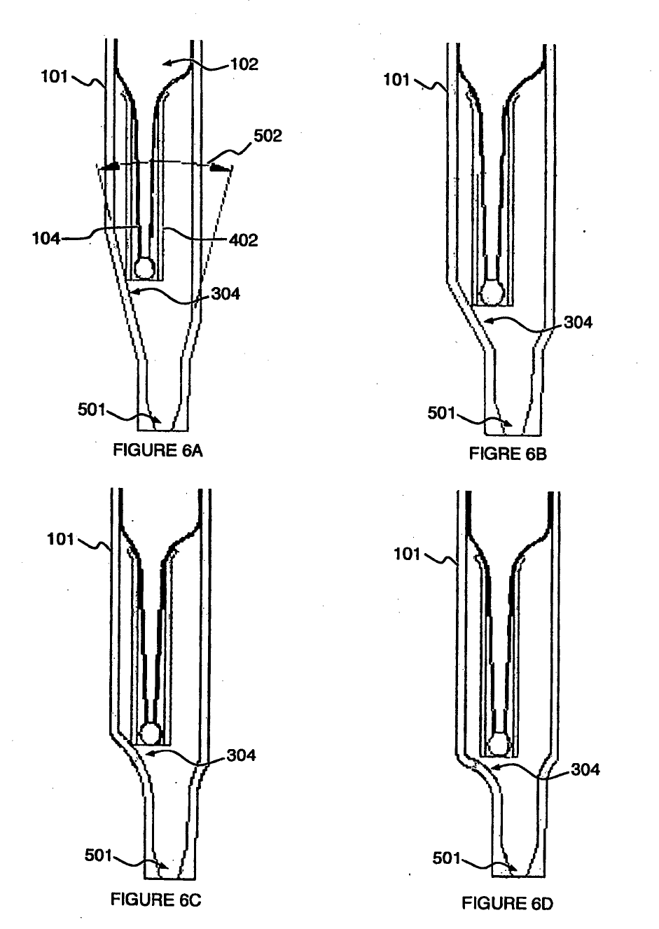

Referring to FIGS. 6A-D, a bottom port 501 of the ampoule barrel 101 is offset, for example, by 0.065 inches from a centerline of the ampoule barrel 101. Referring to FIG. 6A, a lead in angle 502 of the face 304 is about 30 degrees. A height of the face is not uniform around the ampoule barrel 101. The rotation of the ampoule 102 within the ampoule barrel 101 ensures that the tip 104 contacts the face 304 upon descending into the ampoule barrel 101. The rotation may be adjusted manually. A mechanism, such as a cooperating shape of the ampoule barrel 101 and ampoule 102, may secure an alignment A sheath 402 may be implemented as a guard over the tip 104. Referring to FIG. 6B, a lead in angle of the face 304 is about 60 degrees. Referring to FIGS. 6C and 6D a face 304 has a radius of about 0.500 inches and 0.250 inches, respectively.

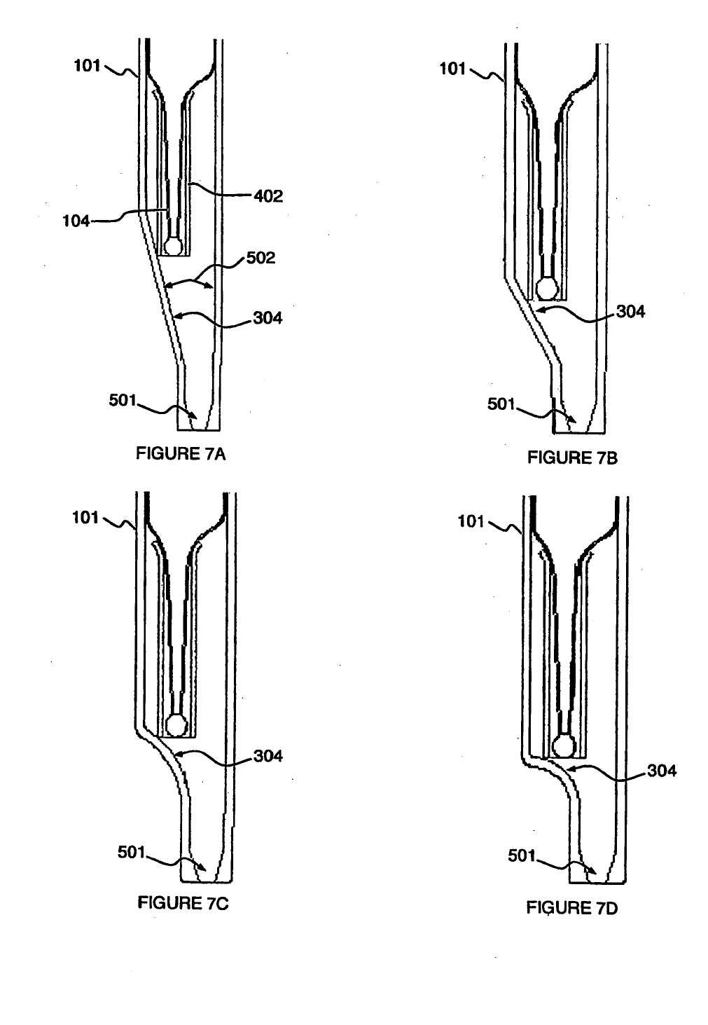

Referring to FIGS. 7A-D, a bottom port 501 of the ampoule barrel 101 is offset, for example, by 0.130 inches from a centerline of the ampoule barrel 101. Referring to FIG. 7A, a lead in angle 502 of the face 304 is about 30 degrees. The angle of the face varies around the circumference of the ampoule barrel 101 between about 90 degrees and about 75 degrees. A height of the face is not uniform around the ampoule barrel 101. The rotation of the ampoule 102 within the ampoule barrel 101 ensures that the tip 104 contacts the face 304 upon descending into the ampoule barrel 101. Referring to FIG. 7B, a lead in angle of the face 304 is about 60 degrees. Referring to FIGS. 7C and 7D a face 304 has a radius of about 0.500 inches and 0.250 inches, respectively. A sheath 402 may be implemented as a guard over the tip 104.

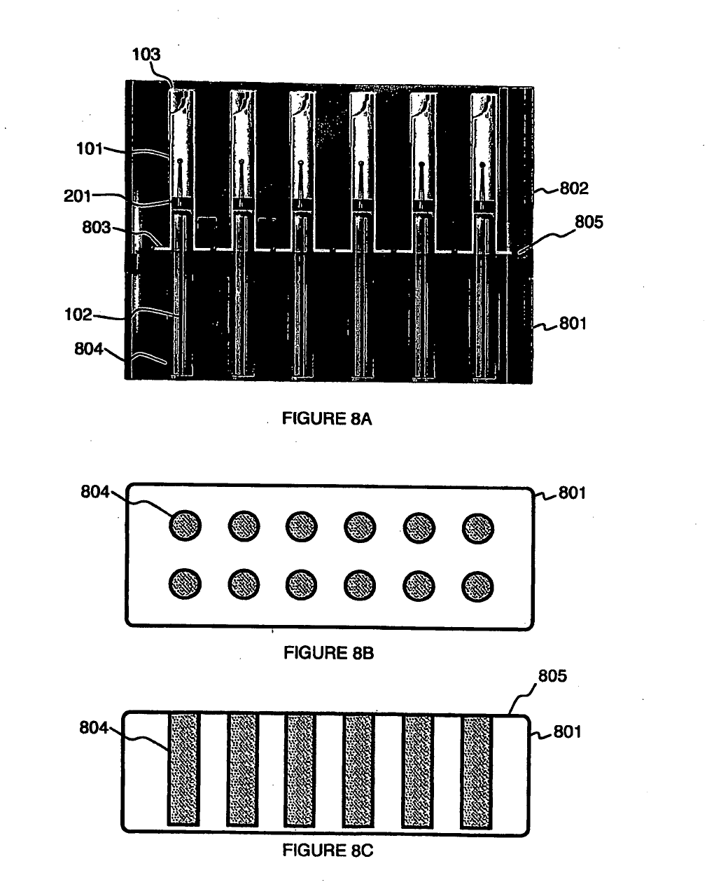

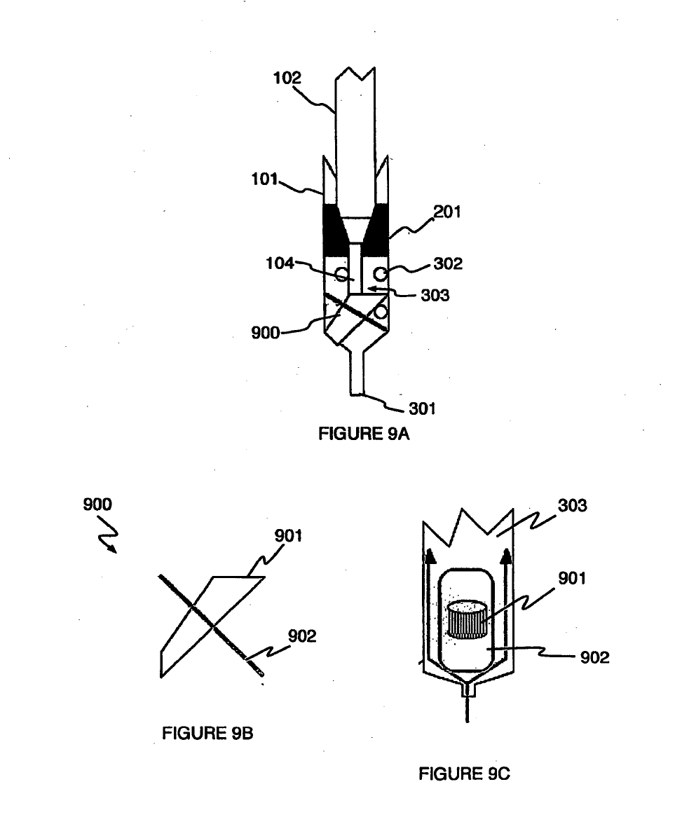

Referring to FIGS. 8A-C, a test system 100 may be loaded into a carrier 801. The carrier 801 may be capped by a top 802. The carrier comprises one or more trenches 804 for receiving a portion of an ampoule 102. The trench 804 has a depth adapted to support an unused system 100 such that the ampoule 102 is not pressed into the ampoule barrel 101; a distance between a bottom of the trench and the upper surface 805 prevents the descent of the ampoule into the ampoule barrel. A flange 803 of the ampoule barrel 101 rests on an upper surface of the carrier 801.

An ampoule 102 according to an embodiment of the present disclosure is a sterile vacuum packaging ampoule containing a dry, non-hazardous, test reagent system. The ampoule 102 prevents user contamination or hazard, has about a 4-year product shelf life, does not trigger transportation restrictions and does not need climate-controlled storage.

A test of a liquid may be performed using a test system 100 according to an embodiment of the present disclosure. A sample module or ampoule barrel 101 secures a test ampoule 102 for extracting a liquid sample. The ampoule barrel 101 limits a sample taker's exposure to the liquid. When used with a pre-dosed test ampoule 102, the ampoule barrel 101 and test ampoule 102 automatically start a test of the sampled liquid under the pressure of the vacuum.

Referring to FIG. 9A, the sample chamber 303 houses an ampoule tip breaker assembly 900. The ports or inlets, e.g., 301 and 302, may be formed on the walls of the ampoule barrel 101 and at a tip of the ampoule barrel 101. Any number of inlets may be used. The inlets allow the sample to freely enter the sample chamber at a rate at least as great as the sample enters the ampoule 102. Thus, the sample may enter the ampoule 102 in a predetermined dose, substantially unaffected by suction or fluid resistance. The ampoule tip breaker assembly 900 is stabilized in the sample chamber 303 to receive a tip 104 of the ampoule 102.

Referring to FIGS. 9B and 9C, the ampoule tip breaker assembly 900 is formed such that the sample may flow around the assembly and enter the ampoule 102. The ampoule tip breaker assembly 900 includes a surface disposed at an angle for breaking the frangible portion of the test ampoule. The surface may be a hollow tube 901 for receiving a tip 104 of an ampoule 102 and for breaking the tip 104. The hollow tube 901 is disposed at an angle to apply a substantially lateral force against the tip to facilitate the breaking of the tip. 104 For example, the hollow tube may be disposed at about 45 degrees from the walls of the sample chamber. The assembly 900 includes a stabilizer support 902 disposed at an angle for supporting the hollow tube 901, e.g., at about 90 degrees from the angle of the hollow tube 901. The stabilizer support 302 and the hollow tube 901 may be formed as one piece. The hollow tube 901 may collect a broken portion of the tip of the ampoule 102 upon breaking away from the ampoule 102.

The stabilized ampoule tip breaker 900 breaks the tip of the ampoule 102 upon the application of pressure to the ampoule 102, forcing the test ampoule tip 104 to engage a surface disposed at an angle. The tip 104 of the ampoule 102 is submerged in liquid as to avoid suction entrained air and creating an unacceptable ampoule fill.

The test ampoule may be a hard-surfaced, self-filling container. The test ampoule includes mixed test indicators/media in predetermined quantities for performing a complete microbiological test. The test ampoules may be sealed, having a vacuum of about 20-30 inches of mercury or more. The test ampoule and contents may be insensitivity to storage conditions and may have a shelf life of about 4 years or more. The test ampoule includes a frangible area that can be broken, allowing a predetermined amount of sample to enter the test ampoule and be exposed to the test indicators/media.

The test ampoule may be an ampoule as described in U.S. Pat. Nos. 5,159,799 entitled VIAL WITH POWDERED REAGENT, 5,550,032 entitled BIOLOGICAL ASSAY FOR MICROBIAL CONTAMINATION, and 5,935,799 entitled BIOLOGICAL ASSAY FOR MICROBIAL CONTAMINATION, each patent being incorporated herein by reference in the entirety.

A test ampoule may be a pre-dosed, hermetically sealed, vacuum ampoule. The vacuum packaging of test ampoule preserves the reagent/media for years and needs no special storage conditions such as refrigeration. When the test is started, a aqueous sample of a predetermined volume, e.g., 7.5 ml, is automatically drawn into the test ampoule. The volume of sample drawn can be any predetermined amount, depending on, for example, the size of the test ampoule and the strength of the vacuum. The test may be concluded when the test ampoule turns a predetermined color, e.g., orange or red. The elapsed time from test start to test end determines the level of microbial contamination. Test results may come as fast as one (1) hour for concentrations of 201 or twelve (12) hours for 10¹ microbial concentrations. The test ampoule may be used as presence/absence test at 24 hours. A Triphenyltetrazoliumchloride (TTC) indicator may react to aerobic microbial activity in the sample to include facultative species. Fungi may also be detected. The presence of fungi may be indicated by floating red particles after 24 hours. Time/Concentration calibrations are based upon mixed microbial populations typically found in industrial and natural waters. Waters dominated by a particular species may use a one-time calibration adjustment. Each test ampoule comes complete with a sample/ampoule, snapping cup, dechlorinating solution, sample identification labels, waste-water instructions and a results/instruction chart Test incubation temperature can be controlled, and may be set to, for example, 95° F. or room temperature. Test incubation can be performed manually by purchasing a reusable carry incubation tube or using a standard laboratory heat block or oven. Automatic incubation and end of test detection can accomplished using an incubator/auto-analyzer. Factory-prepared test calibrations/formulations and/or private labeling may also to used.

An insulated chamber, such as an autoincubation chamber, suitable to hold a plurality of test ampoules at a controlled temperature and for specific time initiates and maintains an incubation temperature for a period of time and may return to refrigeration. This chamber may be transportable for all operational phases of the test (refrigeration to incubation back to refrigeration). The test ampoule, sample module, and insulated chamber may be pre-assembled into a clean or sterilized product that is operated by the sample technician or test initiator.

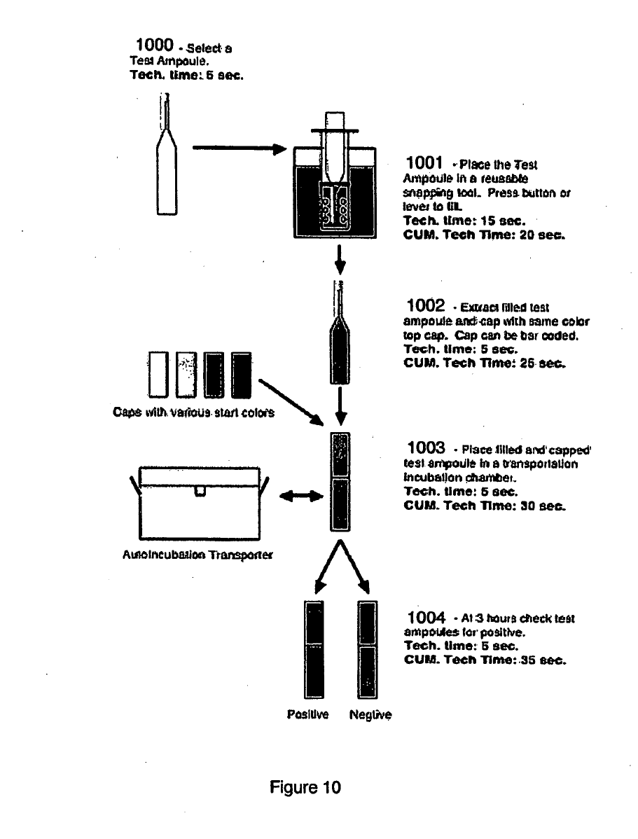

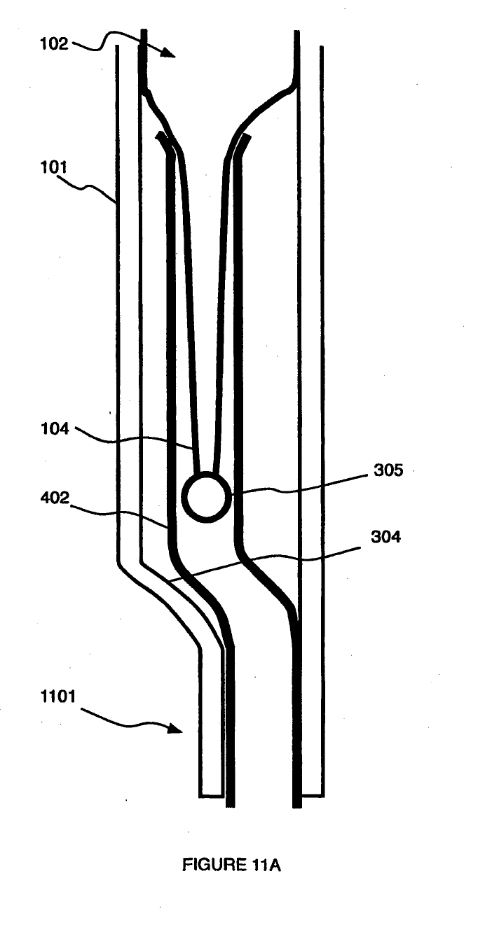

Referring to FIG. 10, an ampoule for a desired test is selected 1000. The ampoule is placed in an ampoule barrel. The ampoule barrel is at least partially immersed in a liquid sample, and the tip of the ampoule is broken to begin a test 1001. The ampoule containing a sample is extracted from the sample module 1002. A cap may be placed over the broken tip of the test ampoule. The cap may include, for example, a bar code for tracking and/or a color chart for determining results. The test ampoule may be placed in an autoincubation testing chamber 1003. The autoincubation testing chamber may be designed for shipping to a laboratory or other location, wherein the autoincubation testing chamber may be coupled to a power source. A control device of the autoincubation testing chamber controls a temperature profile (e.g., heating or cooling of samples under test). The control device may include a processor for outputting control signals to a heater or chiller, and memory device for storing, for example, temperature and time settings. The results may be checked at a predetermined end time 1004. The timing shown in FIG. 10 are provide as examples, actual times may differ depending on the test and procedures.

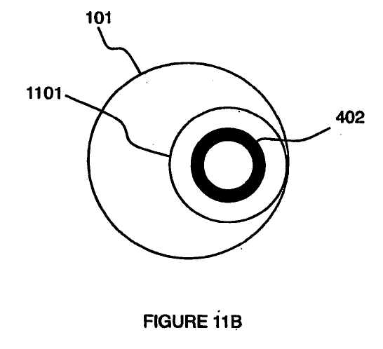

Referring to FIGS. 11A-B, the ampoule barrel 101 includes an offset tip 201. A sheath 402 is fitted over a tip 104 of the ampoule 102. The sheath 402 is formed of a rubberized polyvinyl chloride, a silicon compound or the like. The sheath 402 is press fit on the tip 104 of the ampoule 102. The sheath 402 extends from the tip 104 through the offset tip 101 of the ampoule barrel 101.

The sheath 402 couples the ampoule 102 to the ampoule barrel 101 through the adhesion of the sheath 402 to the ampoule 102 and ampoule barrel 101. The ampoule 102 and ampoule barrel 101 shown in FIG. 11A may be implemented with a cap 103, wherein the cap 103 substantially prevents the ampoule 102 from descending into the ampoule barrel 101. The cap 103 blocks the sheath 402 and the ampoule 102 from being descending into the ampoule barrel 101.

The ampoule 102 may be depressed into the ampoule barrel 101, wherein a terminus 305 meets an angled face 304 of the ampoule barrel 101. A pressure applied to the ampoule 102 causes the ampoule to descend into the ampoule barrel 101. The angled face 304 converts the pressure into a lateral pressure on the terminus 305 of the tip 104. The tip 104 breaks under the lateral pressure. The terminus 305 breaks away from the tip 104 allowing a liquid in which the offset tip 201 is submerged. The terminus 305 is held within the sheath 402 while allowing the liquid to pass. Further, the sheath 402 guards a remaining portion of the tip 104 within the sheath 402.

Having described embodiments for apparatus and method for sampling a liquid, it is noted that modifications and variations can be made by persons skilled in the art in light of the above teachings. It is therefore to be understood that changes may be made in the particular embodiments of the invention disclosed.

What is claimed is:

1. A sampling apparatus comprising:
an ampoule;
an ampoule barrel for receiving the ampoule through a first opening; and
a sheath fitted over a tip of the ampoule and extending from the ampoule and through a second opening of the ampoule barrel.

2. The sampling apparatus of claim 1, wherein the second opening of the ampoule barrel is offset with respect to a center of the ampoule barrel.

3. The sampling apparatus of claims 1, wherein the sheath couples the ampoule to the ampoule barrel through adhesion of the sheath to the ampoule and ampoule barrel.

4. The sampling apparatus of claim 1, wherein the sheath is adapted to flow a liquid into the ampoule.

5. The sampling apparatus of claim 1, wherein the ampoule comprises an inner surface adapted break a frangible tip of the ampoule.

6. The sampling apparatus of claim 5, wherein the ampoule is descendable into the ampoule barrel, wherein a terminus of the ampoule meets the inner surface of the ampoule barrel.

7. The sample apparatus of claim 7, wherein the terminus of the ampoule is a frangible tip, the frangible tip breakable under a lateral pressure applied against the inner surface, and wherein the sheath retains the terminus.

8. The sampling apparatus of claim 1, further comprising a cap coupled to the ampoule barrel and substantially preventing the ampoule from descending into the ampoule barrel.

9. The sampling apparatus of claim 8, wherein the sheath contacts the cap, the cap substantially preventing the sheath from descending through the second opening.
